(19) United States
(12) Patent Application Publication
(10) Pub. No.: US 2021/0350179 A1
(43) Pub. Date: Nov. 11, 2021

(54) METHOD FOR DETECTING ADVERSE CARDIAC EVENTS

(71) Applicant: IMPERIAL COLLEGE OF SCIENCE, TECHNOLOGY AND MEDICINE, London, Greater London (GB)

(72) Inventors: Ghalib A. Bello, London, Greater London (GB); Carlo Biffi, London, Greater London (GB); Jinming Duan, London, Greater London (GB); Timothy J.W. Dawes, London, Greater London (GB); Daniel Rueckert, London, Greater London (GB); Declan P. O'Regan, London, Greater London (GB)

(73) Assignee: IMPERIAL COLLEGE OF SCIENCE, TECHNOLOGY AND MEDICINE, London, Greater London (GB)

(21) Appl. No.: 17/282,631

(22) PCT Filed: Oct. 7, 2019
(86) PCT No.: PCT/GB2019/052819

§ 371 Date: Apr. 2, 2021

(30) Foreign Application Priority Data
Oct. 5, 2018 (GB) 1816281.8

(51) Int. Cl. G06K 9/62; G06N 3/08; G06N 3/04; G06T 7/00

(52) U.S. Cl. CPC G06K 9/6257; G06N 3/08; G06T 2207/30048; G06T 7/0016; G06N 3/04

(57) ABSTRACT

A method (1) is described for training a machine learning model (2) to receive as input a time-resolved three-dimensional model (4) of a heart or a portion of a heart, and to output (3) a predicted time-to-event or a measure of risk for an adverse cardiac event. The method includes receiving a training set (5). The training set (5) includes a number of time-resolved three-dimensional models (4₁, . . . , 4_{N}) of a heart or a portion of a heart. The training set (5) also includes, for each time-resolved three-dimensional model (4₁, . . . , 4_{N}), corresponding outcome data (7₁, . . . , 7_{N}) associated with the time-resolved three-dimensional model (4₁, . . . , 4_{N}). The method (1) of training a machine learning model (2) also includes, using the training set (5) as input, training the machine learning model (2) to recognise latent representations (12) of cardiac motion which are predictive of an adverse cardiac event. The method (1) of training a machine learning model (2) also includes storing the trained machine learning model (2).

### FIELD OF THE INVENTION

The present invention relates to methods of training a machine learning model to learn latent representations of cardiac motion which are predictive of an adverse cardiac event. The present invention also relates to applying the trained machine learning model to estimate a predicted time-to-event or a measure of risk for an adverse cardiac event.

### BACKGROUND

Motion analysis is used in computer vision to understand the behaviour of moving objects in sequences of images. Techniques for vision-based motion analysis aim to understand the behaviour of moving objects in image sequences. In this domain deep learning architectures have achieved a wide range of competencies for object tracking, action recognition, and semantic segmentation.

The traditional paradigm of epidemiological research is to draw insight from large-scale clinical studies through linear regression modelling of conventional explanatory variables. However, this approach does not embrace the dynamic physiological complexity of heart disease. Even objective quantification of heart function by conventional analysis of cardiac imaging has conventionally relied on crude measures of global contraction that are only moderately reproducible and insensitive to the underlying disturbances of cardiovascular physiology. Integrative approaches to risk classification have used unsupervised clustering of broad clinical variables to identify heart failure patients with distinct risk profiles, while supervised machine learning algorithms can diagnose, risk stratify and predict adverse events from health record data. In the wider health domain deep learning has achieved successes in forecasting survival from high-dimensional inputs such as cancer genomic profiles and gene expression data, and in formulating personalised treatment recommendations.

With the exception of natural image tasks, such as classification of skin lesions, biomedical imaging poses a number of challenges for machine learning as the datasets are often of limited scale, inconsistently annotated, and typically high-dimensional. Architectures predominantly based on convolutional neural nets (CNNs), often using data augmentation strategies, have been successfully applied in computer vision tasks to enhance clinical images, segment organs and classify lesions. Segmentation of cardiac images in the time domain is an established visual correspondence task.

Motion analysis has been applied to cardiac systems. For example, US 2012/078097 A1 describes computerized characterization of cardiac wall motion. Quantities for cardiac wall motion are determined from a four-dimensional (i.e., three-dimensional plus time) sequence of ultrasound data. A processor automatically processes the volume data to locate the cardiac wall through the sequence and calculate the quantities from the cardiac wall position or motion. Various machine learning methods are used for locating and tracking the cardiac wall.

WO 2005/081168 A2 describes computer-aided diagnosis systems and applications for cardiac imaging. The computer-aided diagnosis systems implement methods to automatically extract and analyze features from a collection of patient information (including image data and/or non-image data) of a subject patient, to provide decision support for various aspects of physician workflow including, for example, automated assessment of regional myocardial function through wall motion analysis, automated diagnosis of heart diseases and conditions such as cardiomyopathy, coronary artery disease and other heart-related medical conditions, and other automated decision support functions. The computer-aided diagnosis systems implement machine-learning techniques that use a set of training data obtained (learned) from a database of labelled patient cases in one or more relevant clinical domains and/or expert interpretations of such data to enable the computer-aided diagnosis systems to “learn” to analyze patient data.

Deep learning methods have also been applied to analysis and classification tasks in other areas of medicine, for example, Shakeri et al “Deep Spectral-Based Shape Features for Alzheimer's Disease Classification”, Spectral and Shape Analysis in Medical Imaging, First International Workshop, SeSAMI 2016, Held in Conjunction with MICCAI 2016, Athens, Greece, Oct. 21, 2016, DOI: 10.1007/978-3-319-51237-2 2. This article describes classifying Alzheimer's patients from normal subjects using a convolutional neural network including a variational auto-encoder and a multi-layer Perceptron.

### SUMMARY

According to a first aspect of the invention there is provided a method of training a machine learning model to receive as input a time-resolved three-dimensional model of a heart or a portion of a heart, and to output a predicted time-to-event or a measure of risk for an adverse cardiac event. The method includes receiving a training set. The training set includes a number of time-resolved three-dimensional models of a heart or a portion of a heart. The training set also includes, for each time-resolved three-dimensional model, corresponding outcome data associated with the time-resolved three-dimensional model. The method of training a machine learning model also includes, using the training set as input, training the machine learning model to recognise latent representations of cardiac motion which are predictive of an adverse cardiac event. The method of training a machine learning model also includes storing the trained machine learning model.

The training set may include or be derived from magnetic resonance imaging data. The training set may include or be derived from ultrasound data. The training set may include or be derived from multiple types of image data. Outcome data may indicate the timing and nature of any adverse cardiac events associated with a time-resolved three-dimensional model. An adverse cardiac event may include death from heart disease. An adverse cardiac event may include death from any cause. Storing the trained machine learning model may include temporary storage using a volatile storage medium.

Each time-resolved three-dimensional model may include a plurality of vertices. Each vertex may include a coordinate for each of a number of time points. Each time-resolved three-dimensional model may be input to the machine learning model as an input vector which includes, for each vertex, the relative displacement of the vertex at each time point after an initial time point. The vertices of the time-resolved three-dimensional models may be co-registered. In other words, there may be a spatial correspondence between the positions of the vertices in each time-resolved three-dimensional model.

The time-resolved three-dimensional models may all have an equal number of vertices. For each vertex, the relative displacements for the input vector may be calculated with respect to an initial coordinate of the vertex. The input vector may comprise:

x=(xᵥₖ−xᵥ₁yᵥₖ−yᵥₖ,zᵥₖ−zᵥ₁) for all 1≤b≤Nᵥ,2≤k≤Nₜ

In which x is the input vector, xᵥₖ is the Cartesian x-coordinate of the v^{th} of Nᵥ vertices at the k^{th of N}ₜ time points, yᵥₖ is the Cartesian y-coordinate of the v^{th} of Nᵥ vertices at the k^{th} of Nₜ time points, and zᵥₖ is the Cartesian z-coordinate of the v^{th} of Nᵥ vertices at the k^{th} of Nₜ time points.

The machine learning model may include an encoding layer which encodes latent representations of cardiac motion. The dimensionality of the encoding layer may be a hyperparameter of the machine learning model which may be optimised during training of the machine learning model.

The machine learning model may be configured so that the output predicted time-to-event or measure of risk for an adverse cardiac event is determined using a prediction branch which receives as input the latent representation of cardiac motion encoded by is the encoding layer. The prediction branch may be based on a Cox proportional hazards model.

The machine learning model may include a de-noising autoencoder. The de-noising auto-encoder may be symmetric about a central layer. The central layer may be the encoding layer. The de-noising auto-encoder may comprise a mask configured to apply stochastic noise to the inputs. The mask may be configured to set a predetermined fraction of inputs to the machine learning model to zero, the specific inputs being selected at random. Random may include pseudo-random. The predetermined fraction may be a hyperparameter of the machine learning model which may be optimised during training of the machine learning model.

The machine learning model may be trained according to a hybrid loss function which includes a weighted sum of:
a first contribution determined based on the input time-resolved three-dimensional models and corresponding reconstructed models of cardiac motion, each reconstructed model determined based on the latent representations of cardiac motion encoded by the encoding layer; and
a second contribution determined based on the outcome data and the corresponding outputs of predicted time-to-event or measure of risk for an adverse cardiac event.

The first contribution may be determined based on differences between the input time-resolved three-dimensional models and corresponding reconstructed models of cardiac motion. The second contribution may be determined based on differences between the outcome data and the corresponding outputs of predicted time-to-event or measure of risk for an adverse cardiac event.

The reconstructed model of cardiac motion may be determined using a decoding structure which is symmetric to an encoding structure used to encode latent representations of cardiac motion from the input time-resolved three-dimensional model.

The first contribution may be determined based on a difference between the input to the de-noising autoencoder and a corresponding reconstructed output from the de-noising autoencoder.

The weights of the first and second contributions may each be hyperparameters of the machine learning model which may be optimised during training of the machine learning model.

The hybrid loss function, L_{hybrid}, used to train the machine learning model may be:

$${L}_{hybrid} = α {L}_{r} + γ {L}_{s} \\ {L}_{r} = \frac{1}{N} {\sum }_{n = 1}^{N} {‖ {x}_{n} - ψ ( φ ( {x}_{n} ) ) ‖}^{2}$$

$${L}_{s} = - {\sum }_{n = 1}^{N} {δ}_{n} [ {W}^{′} φ ( {x}_{n} ) - log {\sum }_{j ∈ R ( {t}_{n} )} exp ( {W}^{′} φ ( {x}_{j} ) ) ]$$

In which:
α is a weighting coefficient of the reconstruction loss, Lᵣ,
γ is a weighting coefficient of the prediction loss, Lₛ,
N is sample size, in terms of the number of subjects,
xₙ is the n^{th} of N input vectors to the machine learning model 2,
δₙ is an indicator of the status of the n^{th} of N subjects (0=Alive, 1=Dead),
W′ denotes a (1×dₕ) vector of weights, which when multiplied by the dₕ-dimensional latent code 12, φ(x) yields a single scalar W′φ(xᵢ representing the survival prediction for the n^{th} of N subjects,
ψ(φ(xₙ)) is the reconstructed model 15ₙ for the n^{th} of N subjects, expressed in an equivalent way to the input vector xₙ (and having dimensionality equal to input vector xₙ),
R(tₙ) represents the risk set for the n^{th} of N subjects, i.e. subjects still alive (and thus at risk) at the time the n^{th} of N subjects died or became censored ({j:tⱼ>tₙ}), herein censored refers to the subjects outcome being only partially known because, for example, the patient underwent surgery, and
n and j are summation indices.

The machine learning model may include a hidden layer, the hidden layer having a number of nodes which is optimised during training of the machine learning model. The machine learning model may include two or more hidden layers, each hidden layer having a number of nodes which is optimised during training of the machine learning model. Two or more hidden layers may have an equal number of nodes.

Training the machine learning model may include optimising one or more hyperparameters selected from the group consisting of:
a predetermined fraction of inputs to the machine learning model which are set to zero at random;
a number of nodes included in a hidden layer of the machine learning model;
the dimensionality of an encoding layer which encodes a latent representation of cardiac motion;
weights of the first and second contributions to the hybrid loss function;
a learning rate for training the machine learning model; and
an l₁ regularization penalty used for training the machine learning model.

Optimising one or more hyperparameters may include particle swarm optimisation, or any other suitable process for hyperparameter optimisation.

The machine learning model may be trained to output a predicted time-to-event or a measure of risk for an adverse cardiac event associated with heart dysfunction. Heart dysfunction may take the form of pulmonary hypertension. The machine learning model may be trained to output a predicted time-to-event or a measure of risk for an adverse cardiac event associated with heart dysfunction characterised by left or right ventricular dysfunction. Heart dysfunction may take the form of left or right ventricular failure. Heart dysfunction may take the form of dilated cardiomyopathy.

Each time-resolved three-dimensional model may include at least a representation of a left or right ventricle.

Each time-resolved three-dimensional model may be generated from a sequence of images obtained at different time points, or different points within a cycle of the heart. Each time-resolved three-dimensional model may span at least one cycle of the heart. Each time-resolved three-dimensional model may be generated using a second trained machine learning model. The second trained machine learning model may be a convolutional neural network trained to identify one or more anatomical boundaries and/or features. The second machine learning model may generate segmentations of the plurality of images corresponding to one or more anatomical boundaries and/or features. The second machine learning model may employ image registration to track and correlate one or more anatomical features within the plurality of images.

According to a second aspect of the invention, there is provided a non-transient computer-readable storage medium storing a machine learning model trained according to the method of training a machine learning model.

According to a third aspect of the invention, there is provided a method including receiving a time-resolved three-dimensional model of a heart or a portion of a heart. The method also includes providing the time-resolved three-dimensional model to a trained machine learning model. The trained machine learning model is configured to recognise latent representations of cardiac motion which are predictive of an adverse cardiac event. The method also includes obtaining, as output of the trained machine learning model, a predicted time-to-event or a measure of risk for an adverse cardiac event.

The time-resolved three-dimensional model may be derived from magnetic resonance imaging data. The time-resolved three-dimensional model may be derived from ultrasound data. Each time-resolved three-dimensional model may span at least one cycle of the heart.

The time-resolved three-dimensional model may include a number of vertices. Each vertex may include a coordinate for each of a number of time points. The time-resolved three-dimensional model may be input to the trained machine learning model as an input vector which comprises, for each vertex, the relative displacement of the vertex at each time point after an initial time point.

The vertices of the time-resolved three-dimensional model may be co-registered with a number of time-resolved three-dimensional models which were used to train the machine learning model. In other words, there may be a spatial correspondence between the positions of the vertices in the time-resolved three-dimensional model used as input for the method and the positions of the vertices of each time-resolved three-dimensional model which was used to train the machine learning model.

The trained machine learning model may include an encoding layer configured to encode a latent representation of cardiac motion.

The trained machine learning model may be configured so that the output predicted time-to-event or measure of risk for an adverse cardiac event is determined using a prediction branch which receives as input the latent representation of cardiac motion encoded by the encoding layer.

The machine learning model may also output a reconstructed model of cardiac motion. The reconstructed model of cardiac motion may be determined based on the latent representation of cardiac motion encoded in the encoding layer. The reconstructed model of cardiac motion may be determined using a decoding structure which is symmetric to an encoding structure used to encode the latent representation of cardiac motion from the input time-resolved three-dimensional model.

The trained machine learning model may include a de-noising autoencoder.

The trained machine learning model may be configured to output a predicted time-to-event or a measure of risk for an adverse cardiac event associated with heart dysfunction. Heart dysfunction may take the form of pulmonary hypertension.

The time-resolved three-dimensional model may include at least a representation of a left or right ventricle.

The method may also include obtaining a plurality of images of a heart or a portion of a heart. Each image may correspond to a different time or a different point within a cycle of the heart. The method may also include generating the time-resolved three-dimensional model of the heart or the portion of the heart by processing the plurality of images using a second machine learning model.

The second machine learning model may be a convolutional neural network. The second machine learning model may generate segmentations of the plurality of images corresponding to one or more anatomical boundaries and/or features. The second machine learning model may employ image registration to track and correlate one or more anatomical features within the plurality of images.

The trained machine learning model may be a machine learning model trained according to the method of training a machine learning model (first aspect).

### BRIEF DESCRIPTION OF THE DRAWINGS

Certain embodiments of the present invention will now be described, by way of example, with reference to the accompanying drawings in which:

FIG. 1 illustrates a method of training a machine learning model;

FIG. 2 illustrates a method of using a machine learning model;

FIG. 3A shows examples of automatically segmented cardiac images;

FIG. 3B shows examples of time resolved three-dimensional models;

FIG. 4A shows Kaplan-Meier plots of survival probabilities for subjects in a clinical study, obtained using a conventional parameter model;

FIG. 4B shows Kaplan-Meier plots of survival probabilities for subjects in a clinical study, obtained using an exemplary machine learning model (herein termed the 4Dsurvival network);

FIG. 5A shows a 2-dimensional projection of latent representations 12 of cardiac motion derived and used by the 4Dsurvival network;

FIG. 5B shows saliency maps derived for the 4D survival network;

FIG. 6 is a flow diagram of the clinical study;

FIG. 7 illustrates the architecture of an exemplary second machine learning model for processing image data;

FIG. 8 illustrates the architecture of the 4Dsurvival network

FIG. 9 illustrates automated segmentation of the left and right ventricles in a patient with left ventricular failure; and

FIG. 10 shows a three-dimensional model of the left and right ventricles of a patient with left ventricular failure.

### DETAILED DESCRIPTION OF CERTAIN EMBODIMENTS

In the following, like parts are denoted by like reference numbers. The interpretation of dynamic biological systems requires accurate and precise motion tracking, as well as efficient representations of high-dimensional motion trajectories in order to enable use for prediction and/or risk classification tasks. Such motion information may be important in biological systems which exhibit complex spatio-temporal behaviour in response to stimuli or as a consequence of disease processes. In the present specification, methods are described which provide a generalisable approach for modelling time-to-event outcomes and/or event risk classification from time-resolved three-dimensional model data.

The present specification is concerned with the task of predicting, for a particular subject (also referred to as a patient), a time-to-event for an adverse cardiac event, and/or a measure of risk for an adverse cardiac event. The general methods described in this specification have also been assessed in a clinical study described herein.

The motion dynamics of the beating heart are a complex rhythmic pattern of non-linear trajectories regulated by molecular, electrical and biophysical processes. Heart failure is a disturbance of this coordinated activity characterised by adaptations in cardiac geometry and motion that often leads to impaired organ perfusion.

A major challenge in medical image analysis has been to automatically derive quantitative and clinically-relevant information in patients with disease phenotypes such as, for example, heart failure. The present specification describes methods to solve such problems by training a machine learning model to learn latent representations of cardiac motion which are both robust against noise and also relevant for survival prediction and/or risk estimation.

Method of Training a Machine Learning Model

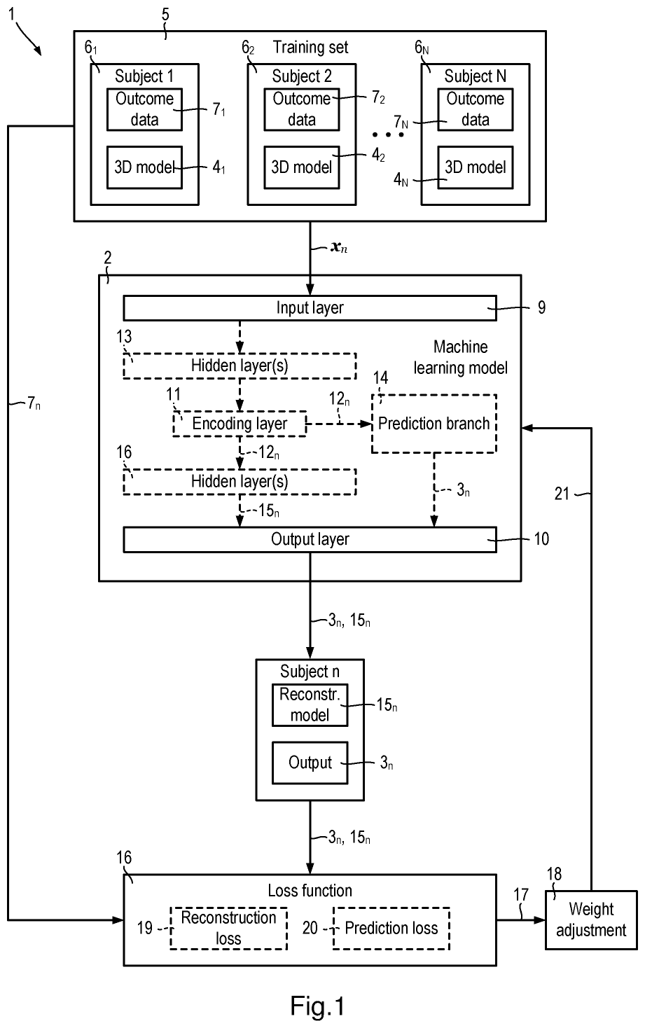

Referring to FIG. 1, a block diagram of a method 1 of training a machine learning model 2 is shown.

The method is used to train the machine learning model 2 to calculate output data 3 in the form of a predicted time-to-event of an adverse cardiac event, and/or a measure of risk for an adverse cardiac event. The machine learning model 2 receives as input a time-resolved three-dimensional model 4 of a heart, or a portion of a heart. An adverse cardiac event may include death from heart disease, heart failure and so forth. An adverse cardiac event may include death from any cause. The adverse cardiac event may be associated with cardiovascular disease and/or heart dysfunction. Cardiovascular disease and/or heart dysfunction may affect one or more of the left ventricle, right ventricle, left atrium, right atrium and/or myocardium. One example of cardiovascular disease is pulmonary hypertension, such as pulmonary hypertension characterised by right and/or left ventricular dysfunction. Another example of cardiovascular disease is left ventricular failure, sometimes also referred to as dilated cardiomyopathy.

The method of training utilises a training set 5. The training set 5 may be either pre-prepared or generated at the point of training, and includes training data 6₁, . . . , 6ₙ, . . . , 6_{N} corresponding to a number, N, of distinct subjects (also referred to as patients). Each subject for whom data 6ₙ is included in the training set 5 has had a scan performed from which a time resolved three-dimensional model 4ₙ has been generated. Each time resolved three-dimensional model 4ₙ may include a representation of the whole or any part of the subject's heart, such as, for example, the right ventricle, left ventricle, right atrium, left atrium, myocardium, and so forth. Each time resolved three-dimensional model 4ₙ may be generated from a sequence of images obtained at different time points, or different points within a cycle of the heart of the n^{th} of N subjects. Each time resolved three-dimensional model 4ₙ may be generated from a sequence of gated images of the subject's heart. A gated image may be built up across a number of heartbeat cycles of the subject's heart, by capturing data from the same relative time within numerous successive heartbeat cycles. For example, gated imaging may be synchronised to electro-cardiogram measurements. Each time-resolved three-dimensional model 4ₙ may span at least one heartbeat cycle of the corresponding subject.

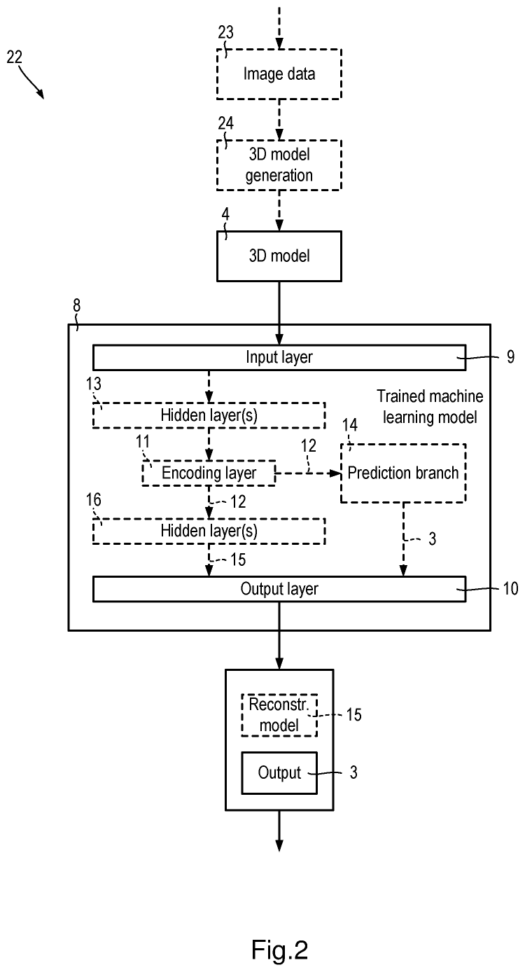

The time resolved three-dimensional models 4₁, . . . , 4ₙ, . . . , 4_{N} included in the training set 5 may include or be derived from magnetic resonance (MR) imaging data. MR imaging data is typically acquired by means of gated imaging. Additionally or alternatively, some or all of the time resolved three-dimensional models 4ₙ, . . . , 4ₙ, . . . , 4_{N} included in the training set 5 may include or be derived from ultrasound data. Although ultrasound data may typically have relatively lower resolution compared to MR imaging data, ultrasound data is easier and quicker to obtain, and the required equipment is significantly less expensive and more portable than an MR imaging scanner. In general, the time resolved three-dimensional models 4₁, . . . , 4ₙ, . . . , 4_{N} included in the training set 5 may be derived from a single type of image data 23 (FIG. 2) or from a variety of types of image data 23 (FIG. 2). The machine learning methods 1, 22 of the present specification are based on latent representations 12ₙ of cardiac motion which are robust against noise, and consequently the machine learning methods 1, 22 merely require that it is possible to acquire the necessary data to produce the time resolved three-dimensional models 4ₙ, . . . , 4ₙ, . . . , 4_{N} used as input. The training data 6ₙ for the n^{th} of N subjects also includes corresponding outcome data 7ₙ for that subject. Outcome data 7ₙ may indicate the timing and nature of any adverse cardiac events associated with the subject, and hence also associated with the corresponding time-resolved three-dimensional model 4ₙ. Outcome data 7ₙ is obtained from long term follow-up of subjects following the scan from which the data for the time-resolved three-dimensional model 4ₙ is obtained. The follow-up period may be as short as a few months, or may be up to several decades, depending on the subject.

According to the method 1, the machine learning model 2 is trained to recognise latent representations 12₁, . . . , 12ₙ, . . . , 12_{N} of cardiac motion which are predictive of either the time to an adverse cardiac event and/or the risks of an adverse cardiac event. Once trained, the machine learning model 2 may be used to encode a latent representation 12 for a new subject, and use the latent representation 12 to calculate output data 3 in the form of a predicted time-to-event of an adverse cardiac event, and/or a measure of risk for an adverse cardiac event.

Once the machine learning model 2 has been trained, for example once the predictive accuracy of the machine learning model 2 when applied to a validation set (not shown) shows no further improvement, the trained machine learning model 2 is stored. For example, when the trained machine learning model 2 (FIG. 2) takes the form of a neural network, the trained machine learning model 2 may be stored by recording the weights of each interconnection between a pair of nodes. In some examples, the numbers of nodes and the connectivity of each node may be varied. In such examples, storing the trained machine learning model 2 may also include storing the number and connectivity of nodes forming one or more layers of the trained machine learning model 2. The validation set (not shown) is structurally identical to the training set 5, except that the time resolved three-dimensional models 4 and outcome data 7 included in the validation set (not shown) correspond to subjects who are not included in the training set 5. The sampling of subjects to form the training set 5 and the validation set (not shown) should be performed at random from the pool of available subjects.

In some examples, a validation set need not be used. This may be the case when the pool of potential subject is small. When a validation set is not used or not available, the predictive accuracy of the machine learning model 2 may be confirmed using a bootstrap internal validation procedure described hereinafter in relation to a clinical study.

Structure of the Machine Learning Model

The machine learning model 2 includes an input layer 9 and an output layer 10. The input layer 9 receives a time-resolved three-dimensional model 4ₙ. Each time-resolved three-dimensional model 4ₙ takes the form of a plurality of vertices Nᵥ. The v^{th} of Nᵥ vertices takes the form of a three-dimensional coordinate, for example, (xᵥ, yᵥ, zᵥ) in Cartesian coordinates. The vertices are mapped to features of the subject's heart to ensure that the same vertex corresponds to the same portion of the subject's heart at each time of the time-resolved three-dimensional model 4ₙ.The time-resolved three-dimensional models may all have an equal number of vertices (xᵥ, yᵥ, zᵥ). The time-resolved three-dimensional models may also include connectivity data defining which vertices are connected to which other vertices to define faces used for rendering the time-resolved three-dimensional model 4ₙ.Although some examples of the machine learning model 2 may additionally make use of such connectivity data, this is not required.

The Nᵥ vertices of the time-resolved three-dimensional models 4₁, . . . , 4ₙ, . . . , 4_{N} may be co-registered. In other words, there may be a spatial correspondence between the position of the Nᵥ vertices in each of the time-resolved three-dimensional model 4₁, . . . , 4ₙ, . . . , 4_{N}. The mapping of vertices to features of subject's hearts may be used to provide such co-registration of vertex locations across different subjects.

The vertex positions (xᵥ, yᵥ, zᵥ) are functions of time, i.e. xᵥ(tₒ+(k−1)δt), yᵥ(tₒ+(k−1)δt), zᵥ(tₒ+(k−1)δt), in which tₒ is an initial time within the heartbeat cycle, for example tₒ=0, and δt is the interval between sampling times for the image sequence used to generate the time-resolved three-dimensional model 4ₙ. A more concise notation for the vertex coordinates is used hereinafter, wherein xᵥₖ=xᵥ(tₒ+(k−1)δt), yᵥₖ=yᵥ(tₒ+(k−1)δt) and zᵥₖ=zᵥ(tₒ+(k−1)δt). Although explained with reference to Cartesian coordinates for convenience, any suitable three-dimensional coordinate system may be used. The total number of sampling times (or gated times) may be denoted Nₜ so that 1≤k≤Nₜ.

Each time-resolved three-dimensional model 4ₙ may be input to the machine learning model 2 as an input vector x which includes, for each vertex (xᵥₖ, yᵥₖ, zᵥₖ), the relative displacement of the vertex (xᵥₖ, yᵥₖ, zᵥₖ) at each time point after an initial time point. For each vertex of a given time-resolved three-dimensional model 4ₙ, the relative displacements for the input vector x may be calculated with respect to an initial coordinate (xᵥ₁, yᵥ₁, zᵥ₁) of the vertex (xᵥₖ, yᵥₖ, zᵥₖ). For example, the input vector x may be formulated as:

x=(xᵥₖ−xᵥ₁,yᵥₖ−yᵥ₁, zᵥₖ−zᵥ₁) for all 1≤v≤Nᵥ,2≤k≤Nₜ (1)

Each time-resolved three-dimensional model 4ₙ is separately converted to a corresponding input vector xₙ, and the time-resolved three-dimensional models 4₁, . . . , 4ₙ, . . . , 4_{N} are processed one at a time or in batches, i.e. sequentially and not in parallel. The input layer 9 includes a number of nodes equal to the length (number of entries) of the input vectors xₙ, and each input vector xₙ in a given training set 5 is of equal length.

The machine learning model may include an encoding layer 11 which encodes a latent representation 12 of cardiac motion. In other words, the machine learning model 2 takes an input vector xₙ corresponding to the n^{th} of N subjects and converts it into the latent representation 12ₙ, which may be encoded in the values of the encoding layer 11. Each latent representation 12ₙ is a dimensionally reduced representation of the same information as the input vector xₙ. Thus, the number of nodes, or dimensionality dₕ, of the encoding layer 11 is less than, preferably significantly less than, the number of nodes, or dimensionality dᵢₙ, of the input layer 9 (equal to the length of xₙ). In some examples, the dimensionality dₕ of the encoding layer 11 may be a hyperparameter of the machine learning model 2, which may be optimised during the method 1 of training the machine learning model 2. The conversion of the input vector xₙ into the latent representation 12 may be performed by one or more encoding hidden layers 13 of the machine learning model 2, connected in order of decreasing dimensionality d (number of nodes) between the input layer 9 and the encoding layer 11.

The machine learning model 2 may be configured so that an output 3ₙ in the form of a predicted time-to-event of an adverse cardiac event, or a measure of risk for an adverse cardiac event, is determined using a prediction branch 14 which receives as input the latent representation 12 of cardiac motion encoded by the encoding layer 11. The prediction branch 14 may be based on a Cox proportional hazards model, or any other suitable predictive model for adverse cardiac events. The output 3ₙ in the form of a predicted time-to-event of an adverse cardiac event, or a measure of risk for an adverse cardiac event, is provided at one or more nodes of the output layer 10.

Additionally, the output layer 10 also provides a reconstructed model 15n of the cardiac motion, which is generated based on the latent representation 12ₙ, for example as encoded by an encoding layer ii. The reconstructed model 15ₙ may be determined from the latent representation 12ₙ by one or more decoding hidden layers 16. The decoding hidden layers 16 may be symmetric with the encoding hidden layers 13, in terms of dimensionality d and connectivity.

In one example, the machine learning model 2 may include hidden layers 13, 16 and an encoding layer 11 which form a de-noising autoencoder. Such a de-noising auto-encoder may be symmetric about the central, encoding layer 11. When the machine learning model 2 includes a de-noising autoencoder, the input layer 9 and/or one or more encoding hidden layers 13 may implement a mask configured to apply stochastic noise to the inputs. For example, the input layer 9 and/or one or more encoding hidden layers 13 may be configured to set a predetermined fraction, f, of entries (i.e. inputs to the machine learning model 2) of each input vector xₙ to zero, the specific entries being selected at random. Herein, the term random encompasses pseudo-random numbers and processes. The predetermined fraction f may be a hyperparameter of the machine learning model 2 which may be optimised during the method 1 of training the machine learning model 2.

Alternatively, the input layer 9 and/or one or more encoding hidden layers 13 may be configured to add a random amount of noise to a predetermined fraction, f, of entries (i.e. inputs to the machine learning model) of each input vector xₙ, and so forth.

Updating the Machine Learning Model

Each time-resolved three-dimensional model 4ₙ in the training set 5 is processed in sequence, and the corresponding output data 3n and reconstructed model 15n are used as input to a loss function 16 for training the machine learning model 2. The loss function provides error(s) 17 (also referred to as discrepancies or losses) to a weight adjustment process 18.

For example, the error 17 may take the form of a hybrid loss function which is a weighted sum of
1. A first contribution in the form of a reconstruction loss 19, determined based on the input time-resolved three-dimensional model 4ₙ and the corresponding reconstructed model 15ₙ of cardiac motion; and
2. A second contribution in the form of a prediction loss 20, determined based on the outcome data 7ₙ obtained by clinical follow-up of the n^{th} subject and the corresponding output data 3ₙ.

The reconstruction loss 19 may be determined based on differences between the input time-resolved three-dimensional model 4ₙ and the corresponding reconstructed model 15ₙ of cardiac motion. In some examples, the prediction loss 20 may be determined based on differences between the outcome data and the corresponding outputs of predicted time-to-event or measure of risk for an adverse cardiac event.

Training the machine learning model 2 based on a loss function 16 having contributions from a reconstruction loss 19 and also a prediction loss 20 may help to ensure that the machine learning model 2 is trained to recognise latent representations 12 which are indicative of the most important geometric/dynamic aspects of a time resolved three-dimensional model 4. Use of a hybrid loss function may help to enforce that said geometric/dynamic aspects are relevant to the prediction task of estimating output data 3 in the form of a predicted time-to-event of an adverse cardiac event, and/or a measure of risk for an adverse cardiac event.

The relative weightings of the reconstruction loss 19 and the prediction loss 20 may each be hyperparameters of the machine learning model 2 which may be optimised during the method 1 of training the machine learning model 2.

In one example, the loss function 16 used to train the machine learning model 2 may take the form of a hybrid loss function, L_{hybrid} according to:

$${L}_{hybrid} = α {L}_{r} + γ {L}_{s} \\ {L}_{r} = \frac{1}{N} {\sum }_{n = 1}^{N} {‖ {x}_{n} - ψ ( φ ( {x}_{n} ) ) ‖}^{2} \\ {L}_{s} = - {\sum }_{n = 1}^{N} {δ}_{n} [ {W}^{′} φ ( {x}_{n} ) - log {\sum }_{j ∈ R ( {t}_{n} )} exp ( {W}^{′} φ ( {x}_{j} ) ) ] \qquad ( 2 )$$

In which:
a is a weighting coefficient of the reconstruction loss, Lᵣ,
y is a weighting coefficient of the prediction loss, Lₛ,
N is sample size, in terms of the number of subjects,
xₙ is the n^{th} of N input vectors to the machine learning model 2,
δₙ is an indicator of the status of the n^{th} of N subjects (0=Alive, 1=Dead),
W′ denotes a (1×dₕ) vector of weights, which when multiplied by the dₕ-dimensional latent code 12, φ(x) yields a single scalar W′φ(xᵢ) representing the survival prediction for the n^{th} of N subjects,
ψ(φ(xₙ)) is the reconstructed model 15ₙ for the n^{th} of N subjects, expressed in an equivalent way to the input vector n^{th} (and having dimensionality equal to input vector xₙ),
R(tₙ) represents the risk set for the n^{th} of N subjects, i.e. subjects still alive (and thus at risk) at the time the n^{th} of N subjects died or became censored ({j:>tⱼ>tₙ}), herein censored refers to the subjects outcome being only partially known because, for example, the patient underwent surgery, and
n and j are summation indices.

The weight adjustment process 18 calculates updated weights/adjustments 21 for each node of the machine learning model 2 and/or connections between the nodes, and updates the machine learning model 2. For example, the updating may utilise back-propagation of errors. The updating of the machine learning model 2 is typically performed using a learning rate to avoid over-fitting to the most recently processed time resolved three-dimensional model 4ₙ.In accordance with common practices, training of the machine learning model 2 may take place across two or more epochs. In some example, the size of the training set 5 may be expanded using suitable data augmentation strategies.

The method 1 of training the machine learning model 2 may include optimising one or more hyperparameters selected from the group of:
a predetermined fraction f of entries in the input vector xₙ which are randomly set to zero, or otherwise modified at random;
a dimensionality d (number of nodes) of one or more hidden layers 13, 16 of the machine learning model 2;
a dimensionality dₕ of the encoding layer 11 which encodes the latent representation 12 of cardiac motion;
weights a, y of the reconstruction loss 19 and/or the prediction loss 20;
a learning rate for training the machine learning model 2; and
an l₁ regularization penalty used for training the machine learning model 2.

Depending upon the structure of machine learning model 2, not all of these hyperparameters will be used in every example of the machine learning model 2. Some examples of the machine learning model 2 may not use any hyperparameters, or may use different hyperparameters to those listed herein. Optimising one or more hyperparameters of the machine learning model 2 may be performed using any suitable technique such as, for example, particle swarm optimisation.

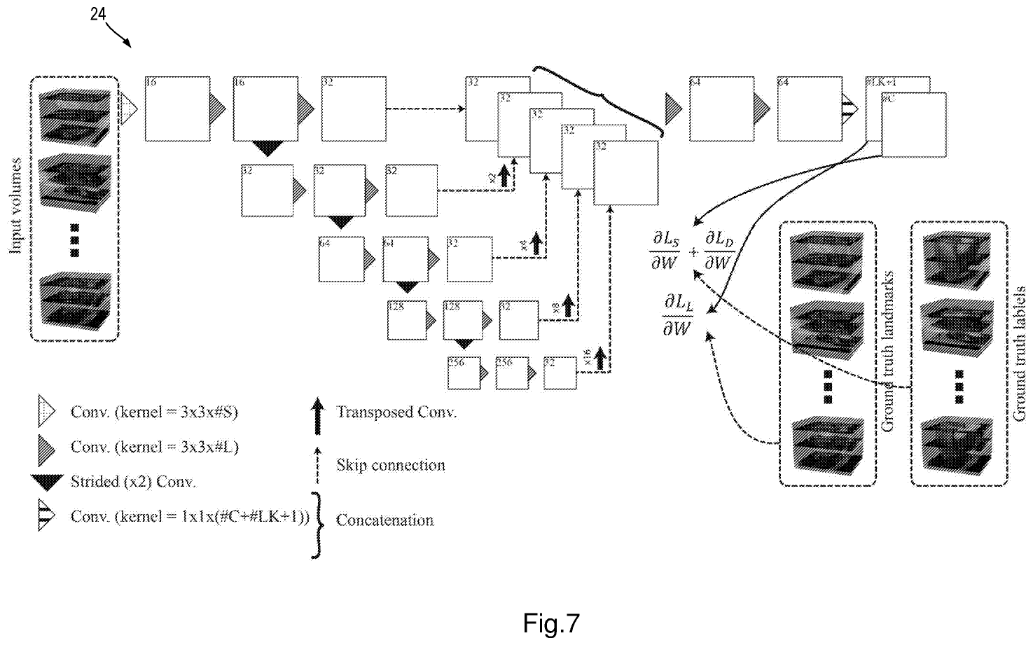

Each of the time resolved three-dimensional models 4₁, . . . , 4ₙ, . . . , 4_{N} may be generated from original image data 23 (FIG. 2) using a second machine learning model 24 (FIGS. 2, 7). The second trained machine learning model 24 (FIGS. 2, 7) may be a convolutional neural network trained to identify one or more anatomical boundaries and/or features of a subject's heart. The second machine learning model 24 (FIGS. 2, 7) may generate segmentations of image date 23 (FIG. 2) in the form of a plurality of images corresponding to one or more anatomical boundaries and/or features of the subject's heart. The second machine learning model 24 (FIGS. 2, 7) may employ image registration to track and correlate one or more anatomical features within the plurality of images. An example of second machine learning model 24 (FIGS. 2, 7) is explained hereinafter.

Once the method 1 is complete, the trained machine learning model 2, or at least the portions of the trained machine learning model 2 necessary for obtaining output data 3 from an input time resolved three-dimensional model 4, may be stored on a non-transient computer-readable storage medium (not shown). For example, when a reconstructed model 15 is not needed in use, it may be considered to store only the input layer 9, the encoding hidden layers 13, the encoding layer ii, the prediction branch 14 and the part of the output layer 10 providing output data 3. However, in practice, the entire machine learning model 2 would typically be stored for convenience and also to allow inspection of the reconstructed models 15 to enable checking that output data 3 has been derived from a sensible latent representation 12. For example, if the reconstructed model 15 does not look like a heart, then the corresponding output data 3 may be regarded as questionable.

Method of Estimating a Predicted Time-to-Event of an Adverse Cardiac Event, and/or a Measure of Risk for an Adverse Cardiac Event

Referring also to FIG. 2, a block diagram of a method 22 of using a machine learning model 2 trained according to the method 1 is shown.

The method 22 includes receiving a time-resolved three-dimensional model 4 of a heart or a portion of a heart, and providing the time-resolved three-dimensional model 4 to the trained machine learning model 2. As explained hereinbefore, the trained machine learning model 2 is configured to recognise latent representations 12 of cardiac motion which are predictive of an adverse cardiac event and/or indicative of a measure of risk for an adverse cardiac event. The method 22 also includes obtaining output data 3 from the trained machine learning model 2 in the form of a predicted time-to-event of an adverse cardiac event, and/or a measure of risk for an adverse cardiac event. The time resolved three-dimensional model 4, the trained machine learning model 2, and the output data 3 are all the same as described in relation to the method 1 of training a machine learning model 2. The trained machine learning model 2 is the product of the method 1 of training a machine learning model 2.

Although not essential, the method 22 may also include obtaining a reconstruction 15 of the input time-resolved three-dimensional model 4. Obtaining the reconstruction 15 may be useful for visualisation purposes, for example to allow inspection of the reconstructed models 15 to check that output data 3 has been derived from a sensible latent representation 12. For example, if the reconstructed model 15 does not look like a heart, then the corresponding output data 3 may be regarded as questionable. Optionally, the method 22 may also include obtaining or receiving image data 23 of a subject's heart, or a portion thereof. The image data 23 may take the form of a sequence of images corresponding to different time points throughout one or more complete cardiac cycles. In general, the image data 23 will include a number of images for each time point, for example, a stack of images for each time point, each image corresponding to a slice through a cross-section of the subject's heart which is offset from each other image. The image data 23 may be obtained using any suitable technique such as, for example, magnetic resonance imaging, ultrasound, and so forth.

The method may also include processing the image data 23 to generate segmented images, then using the segmented images to generate a corresponding time-resolved three-dimensional model 4 of the subject's heart or a portion thereof, using a second machine learning model 24. The second trained machine learning model 24 may be a convolutional neural network trained to identify one or more anatomical boundaries and/or features of a subject's heart. The second machine learning model 24 may generate segmentations of a plurality of images corresponding to one or more anatomical boundaries and/or features of the subject's heart. The second machine learning model 24 may employ image registration to track and correlate one or more anatomical features within the plurality of images. An example of second machine learning model 24 is detailed hereinafter.

Although it has been described to optionally process image data 23 using the second machine learning model 24 in order to generate a time resolved three-dimensional model 4, this is not essential. The trained machine learning model 8 may generate the output data 3 by processing any suitable time resolved three-dimensional model 4, however it is originally obtained.

Experimental Study

The methods 1, 22 of the present specification have been investigated in a clinical study, the results and methods of which shall be described and discussed hereinafter in order to provide relevant context. The clinical study relates to one exemplary implementation of the general methods 1, 22 of the present specification. Although details of the exemplary machine learning model 2 used in the clinical study, termed 4Dsurvival network, provide context and verification of the methods 1, 22, the methods 1, 22 and the appended claims should not be construed as being limited by or to any specific details of the clinical study or the 4Dsurvival network described hereinafter.

The clinical study used image data 23 corresponding to the hearts of 302 subjects (patients), acquired using cardiac magnetic resonance (MR) imaging, to create time-resolved three-dimensional models 4₁, . . . , 4ₙ, . . . , 4_{N}, which were generated using an exemplary second machine learning model 24 in the form of a fully convolutional network trained on anatomical shape priors. The time-resolved three-dimensional models 4₁, . . . , 4ₙ, . . . , 4N so generated formed the input to an exemplary machine learning model 2 in the form of a supervised denoising autoencoder, herein referred to as the 4Dsurvival network, which took the form of a hybrid network including an autoencoder configured to learn a task-specific latent code representations 12 trained on observed outcome data 7₁, . . . , 7ₙ, . . . , 7_{N}. In this way, the trained machine learning model 2, i.e. the trained 4D survival network, was able to generate latent representations 12 optimised for survival prediction.

In order to handle right-censored survival outcomes, the 4D survival network 2 used for the clinical study was trained using a loss function 16 based on a Cox partial likelihood loss function. The clinical study included 302 subject (patients), and the predictive accuracy (quantified by the C-index, see Equation (8)) was significantly higher (p<0.0001) for the 4D survival network 2, with C=0.59 (95% confidence interval, CI: 0.68-0.78), than for a comparison human benchmark of C=0.59 (95% C1: 0.53-0.65). The clinical study provides evidence of how the methods 1, 22 of the present specification may be used to efficiently and accurately predict human survival by estimating a time-to-event for an adverse cardiac event and/or a measure of risk for an adverse cardiac event

For the clinical study, the 302 subjects (patients) studied had been diagnosed with pulmonary hypertension (PH), characterised by right ventricular (RV) dysfunction. This group was chosen as this is a disease with high mortality where the choice of treatment depends on individual risk stratification.

The training set 5 used for the clinical study was derived from cardiac magnetic resonance (CMR), which acquires imaging of the heart in any anatomical plane for dynamic assessment of function. A separate validation set was not used. Instead, a bootstrap internal validation procedure described hereinafter was used. While conventional, explicit measurements of performance obtained from myocardial motion tracking may be used to detect early contractile dysfunction and may act as discriminators of different pathologies, one outcome of the clinical study has been to demonstrate that learned features of complex three-dimensional cardiac motion, as learned by a trained machine learning model 2 in the form of the 4Dsurvival network 2, may provide enhanced prognostic accuracy.

A major challenge for medical image analysis has been to automatically derive quantitative and clinically-relevant information in patients with disease phenotypes. The methods 1, 22 of the present specification provide one solution to such challenges.

An example of a second machine learning model 24 was used, in the form a fully convolutional network (FCN), to learn a cardiac segmentation task from manually-labelled priors. The outputs of the exemplary second machine learning model 24 were time resolved three-dimensional models 4, in the form of smooth 3D renderings of frame-wise cardiac motion. The generated time resolved three-dimensional models 4 were used as part of a training set 5 for training the 4Dsurvival network 2, which took the form of a denoising autoencoder prediction network. The 4Dsurvival network was trained to learn latent representations 12 of cardiac motion which are robust against noise, and also relevant for estimating output data 3 in the form of a predicted time-to-event of an adverse cardiac event in the form of subject death. The performance of the trained 4Dsurvival network (which is only one example of a trained machine learning model 2 according to the present specification) was also compared against a benchmark in the form of conventional human-derived volumetric indices used for survival prediction.

The 4Dsurvival network 2 included an autoencoder. Autoencoding is a dimensionality reduction technique in which an encoder (e.g. encoding hidden layers 13) takes an input (e.g. vector x representing a time resolved three-dimensional model 4) and maps it to a latent representation 12 (lower-dimensional space) which is in turn mapped back to the space of the original input (e.g. reconstructed model 15). The latter step represents an attempt to ‘reconstruct’ the input time resolved three-dimensional model 4 from the compressed (latent) representation 12, and this is done in such a way as to minimise the reconstruction loss 19, i.e. the degree of discrepancy between the input time resolved three-dimensional model 4 and the corresponding reconstructed model 15 (alternatively, between input vector x and a corresponding reconstructed output vector, denoted ψ(φ(xₙ)) and further described hereinafter).

The 4Dsurvival network 2 was based on a denoising autoencoder (DAE), which is a type of autoencoder which aims to extract more robust latent representations 12 by corrupting the input, for example vector x representing a time resolved three-dimensional model 4 with stochastic noise. The denoising autoencoder used in the 4Dsurvival network 2 was augmented with a prediction branch 14, in order to allow training the 4Dsurvival network 2 to learn latent representations 12 which are both reconstructive and discriminative. A loss function 16 was used in the form of a hybrid loss function having a contribution from a reconstruction loss 19 and a contribution from a prediction loss 20. The prediction loss 20 for training the exemplary machine learning model 2 was inspired by the Cox proportional hazards model. A hybrid loss function 16, L_{hybrid} was used in order to permit optimisation of the trade-off between accuracy of the output data 3 and accuracy of the reconstructed model 15, and the balance between these aspects was calibrated during training by adjusting the relative weightings α, γ of the contributions 19, 20 to the overall loss function 16. As described hereinafter, the output data 3 from the 4Dsurvival network 2, based on latent representations 12 of cardiac motion, may be observed to predict survival more accurately than a composite measure of conventional manually-derived parameters measured on the same image data 23. To safeguard against overfitting on the training set 5, dropout and L₁ regularization were used in order to yield a robust prediction model.

Baseline Characteristics

Data from all 302 subjects with incident PH were included for analysis. Objective is diagnosis was made according to haemodynamic criteria. Subjects were investigated between 2004 and 2017, and were followed-up until Nov. 27, 2017 (median 371 days). All-cause mortality was 28% (85 of 302). Table 1 summarizes characteristics of the study sample at the date of diagnosis. No subjects' data were excluded.

MR Image Processing

Automatic segmentation of the ventricles from image data 23 in the form of gated CMR images was performed for each slice position at each of 20 temporal phases producing a total of 69,820 label maps for the cohort.

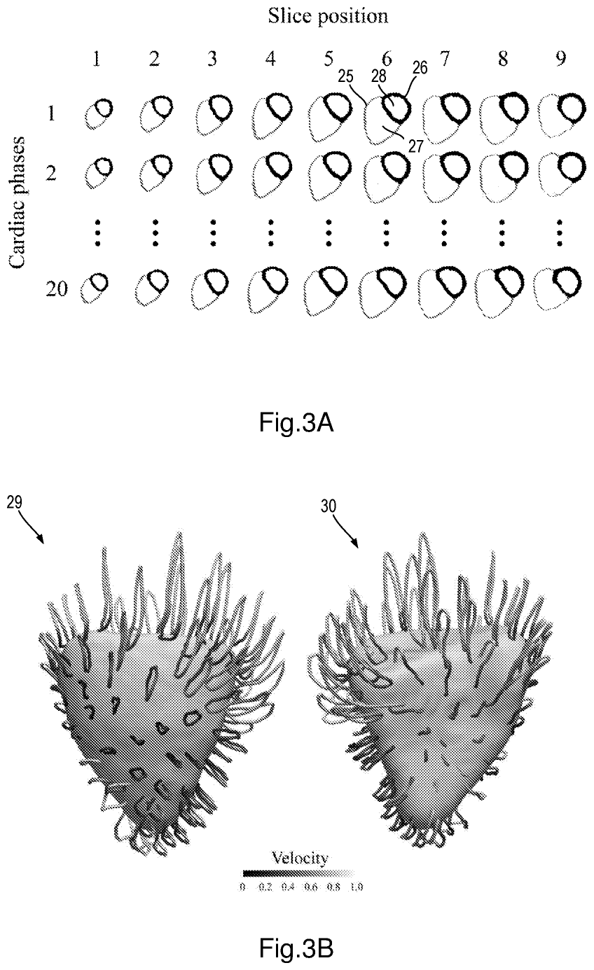

Referring also to FIG. 3A, an example is shown of an automatic cardiac image segmentation of each short-axis cine image from apex (slice 1) to base (slice 9) across 20 time points.

Data were aligned to a common reference space to build a population model of cardiac motion. In each image, the right ventricular wall 25, the left ventricular wall 26, the right ventricular blood pool 27 and the left ventricular blood pool 28 may be observed to have been clearly segmented.

Image registration was used to track the motion of corresponding anatomic points. Segmented image data 23 for each subject was aligned producing a dense time resolved three-dimensional model 4 of cardiac motion, which was then used as an input for training or validating the 4Dsurvival network.

Referring also to FIG. 3B, examples of time resolved three-dimensional models 4 are shown for the freewall 29 and septum 30 of the subject's hearts, averaged across the study population. The time resolved three-dimensional models 29, 30 shown in FIG. 3B were generated by averaging vertex-wise, time-resolved displacement values (along x, y and z coordinates) across all subjects.

Trajectories of right ventricular contraction and relaxation averaged across the study population are also plotted in FIG. 3B as looped pathlines for a sub-sample of 100 points (vertices) on the heart, using a magnification factor of 4 times. The greyscale shading represents relative myocardial velocity at each phase of the cardiac cycle. The surface-shaded models 29, 30 are shown at the end-systole point of a heartbeat cycle. Such dense myocardial motion fields for each subject, for example represented in the form of an input vector x, were used as the inputs to the 4Dsurvival network.

Predictive Performance

Bootstrapped internal validation was applied to the 4Dsurvival network, and also to the benchmark conventional parameter models.

Referring also to Table 1, Patient characteristics are tabulated at baseline (date of MRI scan). The acronyms in Table 1 have the following correspondences: WHO, World Health Organization; BP, Blood pressure; LV, left ventricle; RV, right ventricle.

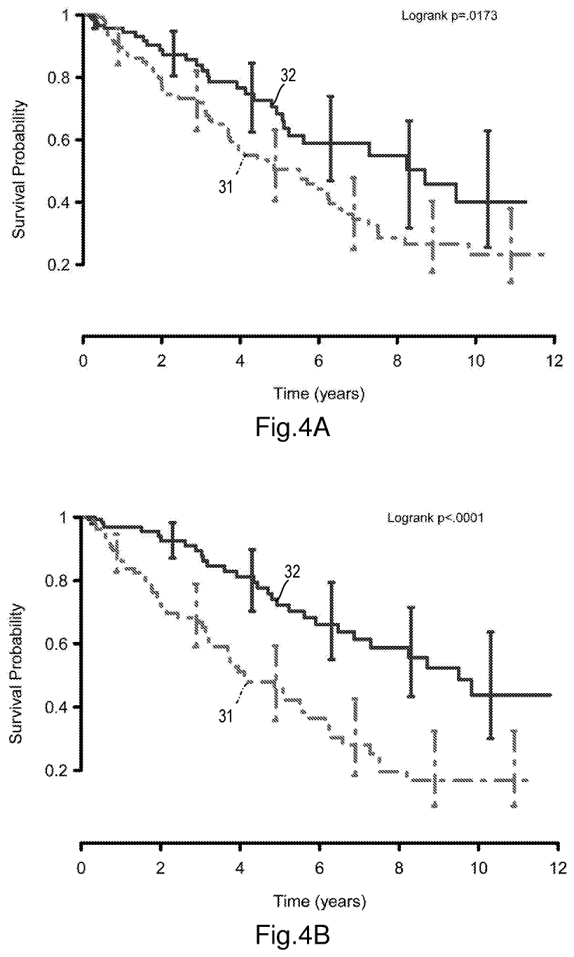

Referring also to FIG. 4A, Kaplan-Meier plots are shown for a conventional parameter model using a composite of manually-derived volumetric measures.

Referring also to FIG.ure 4B, Kaplan-Meier plots are shown for the 4Dsurvival network, using the time resolved three-dimensional models 4 of cardiac motion as input.

For both models, subjects were divided into a low risk group 32 and a high-risk group 31 by median risk score. Survival function estimates for each group 31, 32 (with 95% confidence intervals as error bars) are shown. For the data shown in FIGS. 4A and 4B, the Logrank test was performed to compare survival curves between the risk groups 31, 32. For the conventional parameter model: x²=5.7, p=0.0173; for the 4Dsurvival network: x²=20.7, p<0.0001).

The apparent predictive accuracy for the 4Dsurvival network was C=0.85 and the optimism-corrected value was C=0.73 (95% CI: 0.68-0.78). For the benchmark conventional parameter model, the apparent predictive accuracy was C=0.61 with the corresponding optimism-adjusted value being C=0.59 (95% CI: 0.53-0.65). The accuracy for the 4Dsurvival network was significantly higher than that of the conventional parameter model (p<0.0001). After bootstrap validation, a final model was created using the training and optimization procedure outlined hereinafter, with the Kaplan-Meier plots shown in FIGS. 4A and 4B showing the survival probability estimates over time, stratified by risk groups 31, 32 defined by each model's predictions. Further details of the methods used to validate the 4Dsurvival model are described hereinafter.

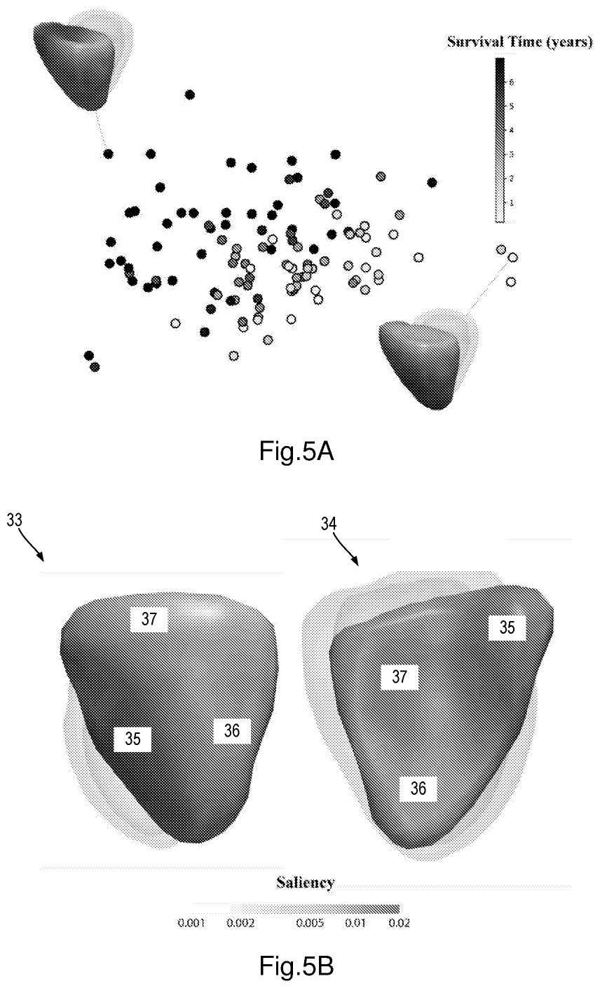

Referring also to FIG. 5A, a 2-dimensional projection is shown of latent representations 12 of cardiac motion derived and used by the 4Dsurvival network. Visualisations of right ventricular motion are also shown for two subjects with contrasting risks.

To assess the ability of the 4Dsurvival network (i.e. one example of a machine learning model 2) to learn discriminative features from the data, the encoded latent representations 12 were examined by projection to 2D space using Laplacian Eigenmaps, as shown in FIG. 5A. In FIG. 5A, each subject is represented by a point, the greyscale shade of which is based on the subject's survival time, i.e. time elapsed from baseline (date of MR imaging scan) to death (for uncensored patients), or to the most recent follow-up date (for censored patients surviving beyond _{7 years).}

Survival time was truncated at 7 years for ease of visualization. As may be observed from FIG. 5A, the 4Dsurvival network's latent representations 12 of cardiac motion show distinct patterns of clustering according to survival time. FIG. 5A also shows visualizations of right ventricular motion for a pair of exemplar subjects at opposite ends of the risk spectrum.

The extent to which motion in various regions of the right ventricle contributed to overall survival prediction was also assessed.

Referring also to FIG. 5B, saliency maps are shown for freewall 33 and septum 34, each showing regional contributions to the survival prediction (output data 3) by right ventricular motion. The greyscale shading corresponds to absolute regression coefficients which are expressed on a log-scale. For each saliency map 33, 34, a region of relatively high saliency 35, a region of relatively low saliency 36, and a region of intermediate saliency 37 are indicated in FIG. 5 for reference.

Fitting univariate linear models to each vertex in the mesh making up a time resolved three-dimensional model 4, the association between the magnitude of cardiac motion and the 4Dsurvival network's predicted risk score was computed, yielding the saliency maps 33, 34 shown in FIG. 5B. It may be observed from the saliency maps 33, 34 that contributions from spatially distant but functionally synergistic regions of the right ventricle may influence survival of subjects suffering from pulmonary hypertension.

Methods of the Clinical Study

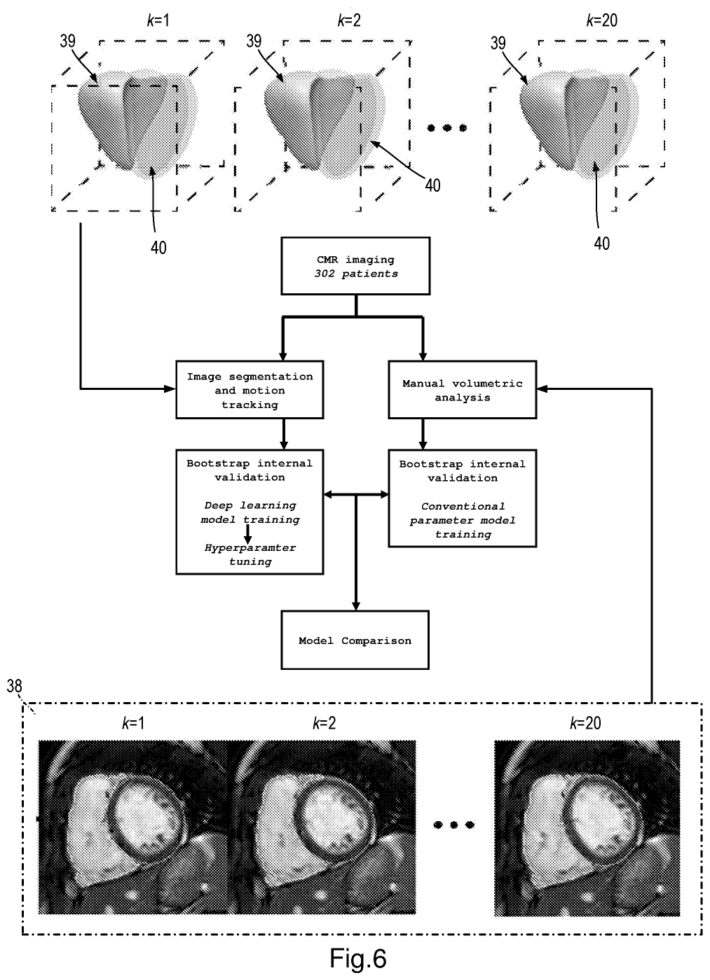

Referring also to FIG. 6, a flowchart of the clinical study is shown.

The clinical study was a single-centre observational study. The analysed data were collected from subjects referred to the National Pulmonary Hypertension Service at the Imperial College Healthcare NHS Trust between May 2004 and October 2017. The study was approved by the Heath Research Authority and all subjects gave written informed consent. Criteria for inclusion were a documented diagnosis of Group 4 pulmonary hypertension investigated by right heart catheterization (RHC) and non-invasive imaging. All subjects were treated in accordance with current guidelines including medical and surgical therapy as clinically indicated.

In total 302 subject had cardiac magnetic resonance imaging, and the corresponding image data 23 was used for both manual volumetric analysis to generate manual segmentations 38, and also for automated image segmentation encompassing the right ventricle 39 and the left ventricle 40, across Nₜ=20 time points (k=1, . . . , 20). Internal validity of the predictive performance of a conventional parameter model and a deep learning motion model was assessed using a bootstrapped internal validation procedure described hereinafter.

MR Image Acquisition, Processing and Computational Image Analysis

Cardiac magnetic resonance imaging was performed on a 1.5T Achieva (Philips, Best, Netherlands), using a standard clinical protocol based on international guidelines. The specific images analysed in the clinical study were retrospectively-gated cine sequences, in the short axis plane of the subject's heart, with a reconstructed spatial resolution of 1.3×1.3×10.0 mm and a typical temporal resolution of 29 ms.

Manual volumetric analysis of the images was independently performed by accredited physicians, according to international guidelines with access to all available images for each subject and no analysis time constraint. The derived parameters included the strongest and most well-established CMR findings for prognostication reported in a disease-specific meta-analysis.

Referring also to FIG. 7, the architecture of an exemplary second machine learning model 24 used for segmenting image data 23 is illustrated.

Briefly, the exemplary second machine learning model 24 took the form of a fully convolutional neural network (CNN), which takes each stack of cine images as an input, applies a branch of convolutions, learns image features from fine to coarse levels, concatenates multi-scale features and finally predicts the segmentation and landmark location probability maps simultaneously. These maps, together with the ground truth landmark locations and label maps, are then used in a loss function which is minimised via back-propagation stochastic gradient descent. Further details of the exemplary second machine learning model 24 used for the clinical study are described hereinafter.

The exemplary second machine learning model 24 was developed as a CNN combined with image registration for shape-based biventricular segmentation of the CMR images forming the image data 23 for each subject. The pipeline method has three main components: segmentation, landmark localisation and shape registration. Firstly, a 2.5D multi-task fully convolutional network (FCN) is trained to effectively and simultaneously learn segmentation maps and landmark locations from manually labelled volumetric CMR images. Secondly, multiple high-resolution three-dimensional atlas shapes are propagated onto the network segmentation to form a smooth segmentation model. This step effectively induces a hard anatomical shape constraint and is fully automatic due to the use of predicted landmarks from the exemplary second machine learning model 24.

The problem of predicting segmentations and landmark locations was treated as a multi-task classification problem. First, the learning problem may be formulated as follows: denoting the input training dataset by S={(Uₙ, Rₙ, Lₙ), n=1, . . . , N }, where N is the sample size of the training data, Uₙ={uⁿₘ, m=1, . . . , |Uₙ|} is the raw input CMR volume for the n^{th} of N subjects, Rₙ={rⁿₘ, m=1, . . . , |Rₙ|}, rⁿₘ∈{1, . . . , Nᵣ} are the ground truth region labels for volume Uₙ(Nᵣ=5 representing 4 regions and background), and Lₙ={lⁿₘ, m=1, . . . , |Lₙ|}, lⁿₘ∈{1, . . . , Nₗ} are the labels representing ground truth landmark locations for Uₙ (Nₗ=7 representing 6 landmark locations and background). Note that |Uₙ|=|Rₙ|=|Lₙ| stands for the total number of voxels in a CMR volume. Let W denote the set of all network layer parameters. In a supervised setting, the following objective function is minimised via standard (backpropagation) stochastic gradient descent (SGD):

L(W)=L_{S}(W)+aL_{d}(W)+bL_{L}(W)+c∥W∥_{F}² (3)

in which a, b and c are weight coefficients balancing the four terms. L_{S}(W) and L_{D}(W) are the region-associated losses that enable the network to predict segmentation maps. L_{L}(W) is the landmark-associated loss for predicting landmark locations. ∥W∥_{F}², known as the weight decay term, represents the Frobenius norm on the weights W. This term is used to prevent the network from overfitting. The training problem is therefore to estimate the parameters W associated with all the convolutional layers. By minimising Equation (3), the exemplary second machine learning model 24 is able to simultaneously predict segmentation maps and landmark locations. The definitions of the loss functions L_{S}(W), L_{D}(W) and L_{L}(W), used for predicting landmarks and segmentation labels, have been described previously, see Duan, J. et al. “Automatic 3D bi-ventricular segmentation of cardiac images by a shape-constrained multi-task deep learning approach.” ArXiv 1808.08578 (2018).

The FCN segmentations are used to perform a non-rigid registration using cardiac atlases built from >1000 high resolution images, allowing shape constraints to be inferred. This approach produces accurate, high-resolution and anatomically smooth segmentation results from input images with low through-slice resolution thus preserving clinically-important global anatomical features. Motion tracking was performed for each subject using a four-dimensional spatio-temporal B-spline image registration method with a sparseness regularisation term. The motion field estimate is represented by a displacement vector at each voxel and at each time frame k=1, . . . , 20. Temporal normalisation was performed before motion estimation to ensure consistency across the cardiac cycle.

Spatial normalisation of each subject's data was achieved by registering the motion fields to a template space. A template image was built by registering the high-resolution atlases at the end-diastolic frame and then computing an average intensity image. In addition, the corresponding ground-truth segmentations for these high-resolution images were averaged to form a segmentation of the template image. A template surface mesh was then reconstructed from its segmentation using a three-dimensional surface reconstruction algorithm. The motion field estimate lies within the reference space of each subject, and so to enable inter-subject comparison all the segmentations were aligned to this template space by non-rigid B-spline image registration. The template mesh was then warped using the resulting non-rigid deformation and mapped back to the template space. Twenty surface meshes, one for each temporal frame, were subsequently generated by applying the estimated motion fields to the warped template mesh accordingly. Consequently, the surface mesh of each subject at each frame contained the same number of vertices (18, 028), which maintained their anatomical correspondence across temporal frames, and across subjects (FIG. 7).

Characterization of Right Ventricular Motion

The time-resolved three-dimensional models 4 generated as described in the previous section were used to produce a relevant representation of cardiac motion—in this example of right-side heart failure limited to the RV. For this purpose, a sparser version of the meshes was utilized (down-sampled by a factor of ˜90) with 202 vertices. Anatomical correspondence was preserved in this process by utilizing the same vertices across all meshes.

This approach was used to produce a simple numerical representation of the trajectory of each vertex, i.e. the path each vertex traces through space during a cardiac cycle (FIG. 3B). The vertex positions (xᵥ, yᵥ, zᵥ) are functions of time, i.e. xᵥ(t₀+(k−1)δt), yᵥ(t₀+(k−1)δt), zᵥ(t₀+(k−1)δt), in which t₀ is an initial time within the heartbeat cycle, for example t₀=0, and δt is the interval between sampling times for the image sequence used to generate the time-resolved three-dimensional model 4ₙ. A more concise notation for the vertex coordinates is used hereinafter wherein xᵥₖ=xᵥ(t₀+(k−1)δt), yᵥₖ=yᵥ(t₀+(k−1)δt) and zᵥₖ=zᵥ(t₀+(k−1)δt). The total number of sampling times may be denoted Nₜ so that 1≤k≤Nₜ. For the clinical study, Nᵥ=202 and Nₜ=20. The input vectors x are formulated according to Equation (1):

x=(xᵥₖ−xᵥ₁,yᵥₖ−yᵥ₁, zᵥₖ−zᵥ₁) for all 1≤v≤Nᵥ,2≤k≤Nₜ (1)

For the data in the clinical study, input vector x has length 11,514 (3×19×202), and was used as input to the 4Dsurvival network.

4Dsurvival Network Design and Training

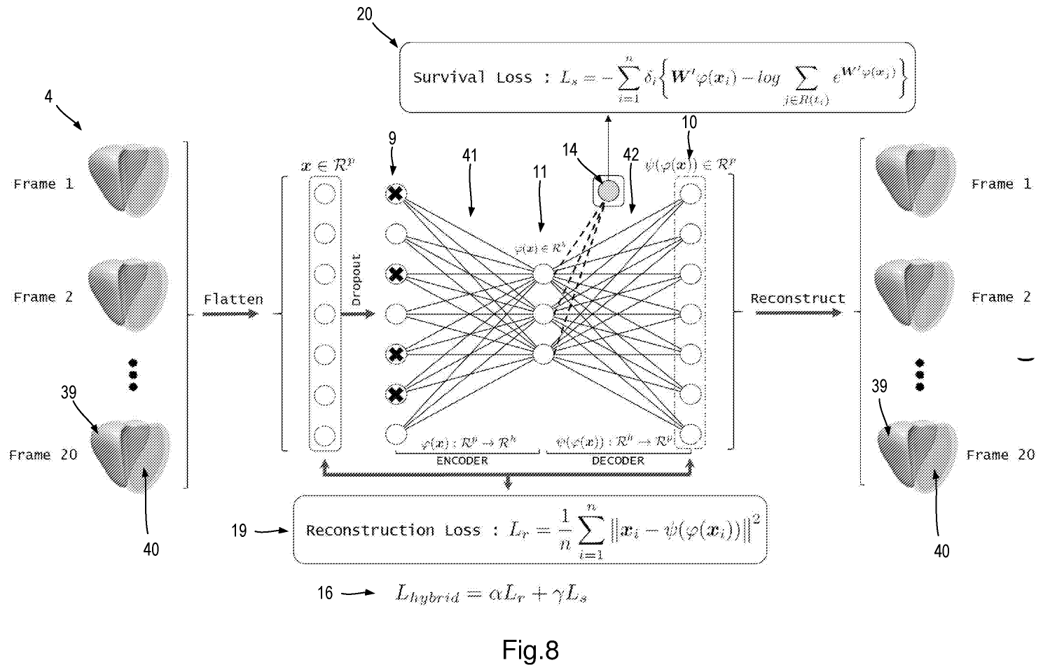

Referring also to FIG. 8, the architecture of the 4Dsurvival network is shown (i.e. one example of a machine learning model 2).

The 4Dsurvival network includes a denoising autoencoder that takes time-resolved three-dimensional models 4 of cardiac motion meshes as its input. The time-resolved is three-dimensional models 4 include representations of the right ventricle 39 and the left ventricle 40. For the sake of simplicity two hidden layers 13, 16, one immediately preceding and the other immediately following the central encoding layer 11, are not shown in FIG. 8. The autoencoder learns a task-specific latent code representation trained on observed outcome data 7, yielding a latent representation 12 optimised for survival prediction that is robust to noise. The actual number of latent factors is treated as an optimisable parameter.

The 4Dsurvival network provides an architecture capable of learning a low-dimensional latent representation 12 of right ventricular motion that robustly captures prognostic features indicative of poor survival. The hybrid design of the 4Dsurvival network combines a denoising autoencoder with an example of a prediction branch 14 which is based on a Cox proportional hazards model (described hereinafter). Again denoting the input vector by x∈ [symbol] ^{d}^{ₚ}, where dₚ=11,514, is the input dimensionality.

The 4Dsurvival network is based on a denoising autoencoder (DAE), an autoencoder variant which learns features robust to noise. The input vector x feeds directly into the encoder 41, the first layer of which is a stochastic masking filter that produces a corrupted version of x. The masking is implemented using random dropout, i.e. a predetermined fraction f of the elements of input vector x were set to zero (the value of f is treated as an optimizable parameter of the 4Dsurvival network). The corrupted input from the masking filter is then fed into a hidden layer 13, the output of which is in turn fed into a central, encoding layer ii. This central, encoding layer 11 represents the latent code, i.e. the encoded/compressed latent representation 12 of the input vector x.

This central encoding layer 11 is sometimes also referred to as the ‘code’, or ‘bottleneck’ layer. Therefore the encoder 41 may be considered as a function φ(.) mapping the input vector x∈ [symbol] ^{d}^{ₚ} to a latent code φ(x)∈ [symbol] ^{d}^{ₕ}, where dₕ [symbol] dₚ (for notational convenience we consider the corruption, or dropout, step as part of the encoder 41). This produces a compressed latent representation 12 having a dimensionality which is lower than that of the input vector x (an undercomplete representation). Note that the number of units in the encoder's hidden layer 13, and the dimensionality dₕ of the latent code are not predetermined but, rather, treated as optimisable parameters of the 4Dsurvival network.

The latent representation 12, φ(x) is then fed into the second component of the denoising autoencoder, a multilayer decoder network 42 that upsamples the code back to the original input dimension dₚ. Like the encoder 41, the decoder 42 has one intermediate hidden layer 16 that feeds into the final, output layer 10, which in turn outputs a decoded representation (with dimension dₚ matching that of the input). In the 4Dsurvival network, this decoded representation corresponds to the reconstructed model 15.

The size of the decoder's 42 intermediate hidden layer 16 is constrained to match that of the encoder 41 networks hidden layer 13, to give the autoencoder a symmetric architecture. Dissimilarity between the original (uncorrupted) input vector x and the decoder's reconstructed model 15 (denoted here by ψ(φ(x))) is penalized by minimizing a loss function of general form L(x, ψ(φ(x))). Herein, a simple mean squared error form is chosen for L:

$${L}_{r} = \frac{1}{N} {\sum }_{n = 1}^{N} {‖ {x}_{n} - ψ ( φ ( {x}_{n} ) ) ‖}^{2} \qquad ( 4 )$$

in which N again represents the sample size in terms of the number of subjects. Minimizing this loss reconstruction loss 19, Lᵣ forces the autoencoder 41, 42 to reconstruct the input x from a corrupted/incomplete version, thereby facilitating the generation of a latent representation 12 with robust features.

As explained hereinbefore, in order to ensure that learned latent representations 12 are actually relevant for estimating output data 3, in this instance in the form of a survival prediction, the autoencoder 41, 42 of the 4Dsurvival network was augmented by adding a prediction branch 14. The latent representation 12 learned by the encoder 41, φ(x) is therefore linked to a linear predictor of survival (see Equation (5)), in addition to the decoder 42. This encourages the latent representation 12, φ(x) to contain features which are simultaneously robust to noisy input and salient for survival prediction. The prediction branch 14 of the 4Dsurvival network is trained with observed outcome data 7, in this instance survival/follow-up time. For each subject, this is time elapsed from MRI acquisition until death (all-cause mortality), or if the subject is still alive, the last date of follow-up. Also, patients receiving surgical interventions were censored at the date of surgery. This type of outcome is called a right-censored time-to-event outcome, and is typically handled using survival analysis techniques, the most popular of which is Cox's proportional hazards regression model:

$$log \frac{{h}_{n} ( t )}{{h}_{o} ( t )} = {β}_{1} {z}_{n 1} + {β}_{2} {z}_{n 2} + ⋯ + {β}_{p} {z}_{n p} \qquad ( 5 )$$

in which hₙ(t) represents the hazard function for subject n, i.e the ‘chance’ (normalized probability) of subject n dying at time t. The term h₀(t) is a baseline hazard level to which all subject-specific hazards hₙ(t) (n=1, . . . , N) are compared. The key assumption of the Cox survival model is that the hazard ratio hₙ(t)/h₀(t) is constant with respect to time (which is termed the proportional hazards assumption). The natural logarithm of this ratio is modelled as a weighted sum of a number of predictor variables (denoted here by zₙ₁, . . . , zₙₚ), where the weights/coefficients are unknown parameters denoted by β₁, . . . , ⊕ₚ. These parameters are estimated via maximization of the Cox proportional hazards partial likelihood function:

$$log L ( β ) = {\sum }_{n = 1}^{N} {δ}_{n} \{ {β}^{′} {z}_{n} - log {\sum }_{j ϵ R ( {t}_{n} )} exp ( {β}^{′} {z}_{j} ) \} \qquad ( 6 )$$

in which, zₙ is the vector of predictor/explanatory variables for subject n, δₙ is an indicator of subject n's status (0=Alive, 1=Dead) and R(tₙ) represents subject n's risk set, i.e. subjects still alive (and thus at risk) at the time subject n died or became censored ({j:tⱼ>tₙ}). This loss function was adapted to provide the prediction loss 20 for the 4Dsurvival network architecture as follows:

$${L}_{s} = - {\sum }_{n = 1}^{N} {δ}_{n} [ {W}^{′} φ ( {x}_{n} ) - log {\sum }_{j ∈ R ( {t}_{n} )} exp ( {W}^{′} φ ( {x}_{j} ) ) ] \qquad ( 7 )$$

The term W′ denotes a (1×dₕ) vector of weights, which when multiplied by the dₕ-dimensional latent code φ(x) yields a single scalar (W′φ(xᵢ)) representing the survival prediction (specifically, natural logarithm of the hazard ratio) for subject n. Note that this makes the prediction branch 14 of the 4Dsurvival network essentially a simple linear Cox proportional hazards model, and the predicted output data 3 may be seen as an estimate of the log hazard ratio (see Equation (5)).

For the 4Dsurvival network, the prediction loss 20 (Equation (7)) is combined with the reconstruction loss 19 (Equation (5)) to form the hybrid loss function 16 of Equation (2), reproduced for convenience:

$${L}_{hybrid} = α {L}_{r} + γ {L}_{s} \\ {L}_{r} = \frac{1}{N} {\sum }_{n = 1}^{N} {‖ {x}_{n} - ψ ( φ ( {x}_{n} ) ) ‖}^{2} \\ {L}_{s} = - {\sum }_{n = 1}^{N} {δ}_{n} [ {W}^{′} φ ( {x}_{n} ) - log {\sum }_{j ∈ R ( {t}_{n} )} exp ( {W}^{′} φ ( {x}_{j} ) ) ] \qquad ( 2 )$$

in which the weighting coefficients α and γ are used to calibrate the contributions of each term 19, 20 to the overall loss function 16, i.e. to control the tradeoff between accuracy of the output data 3 in the form of a survival prediction versus accuracy of the reconstructed model 15. During training of the 4Dsurvival network, the weights α and γ are treated as optimisable network hyperparameters. For the clinical study, γ was chosen to equal (1−α) for convenience.

The loss function 16 was minimized via backpropagation. To avoid overfitting and to encourage sparsity in the encoded representation, we applied L₁ regularization. The rectified linear unit (ReLU) activation function was used for all layers, except the prediction output layer (linear activation was used for this layer). Using the adaptive moment estimation (Adam) algorithm, the 4Dsurvival network was trained for 100 epochs with a batch size of 16 subjects. The learning rate was also treated as a hyperparameter (see Table 2). During training of the 4Dsurvival network, the random dropout (input corruption) was repeated at every backpropagation pass. The entire training process, including hyperparameter optimisation and bootstrap-based internal validation (described hereinafter) took a total of 76 hours.

Hyperparameter Tuning

To determine optimal hyperparameter values, particle swarm optimization (PSO) was used. Particle swarm optimization is a gradient-free meta-heuristic approach for finding optima of a given objective function. Inspired by the social foraging behavior of birds, particle swarm optimization is based on the principle of swarm intelligence, which refers to problem-solving ability that arises from the interactions of simple information-processing units. In the context of hyperparameter tuning, it can be used to maximize the prediction accuracy of a model with respect to a set of potential hyperparameters. Particle swarm optimization was utilised to choose the optimal set of hyperparameters from among predefined ranges of values, summarized in Table 2. The particle swarm optimization algorithm was run for 50 iterations, at each step evaluating candidate hyperparameter configurations using 6-fold cross-validation. The hyperparameters at the final iteration were chosen as the optimal set.

Model Validation and Comparison

Discrimination was evaluated using Harrell's concordance index, an extension of area under the receiver operating characteristic curve (AUC) to censored time-to-event data:

$$C = \frac{{\sum }_{n 1 , n2} {δ}_{n 1} I ( {η}_{n 1} > {η}_{n 2} ) I ( {t}_{n 1} < {t}_{n 2} )}{{\sum }_{n 1 , n2} {δ}_{n 1} I ( {t}_{n 1} < {t}_{n 2} )} \qquad ( 8 )$$

in which the indices n1 and n2 refer to pairs of subjects in the sample and I(·) denotes an indicator function that evaluates to 1 if its argument is true (and o otherwise). Symbols ηₙ₁ and ηₙ₂ denote the predicted risks for subjects n1 and n2. The numerator tallies the number of subject pairs (n1, n2) where the pair member with greater predicted risk has shorter survival, representing agreement (concordance) between the model's risk predictions and ground-truth survival outcomes. Multiplication by δₙ₁ restricts the sum to subject pairs where it is possible to determine who died first (i.e. informative pairs). The C index therefore represents the fraction of informative pairs exhibiting concordance between predictions and outcomes. In this sense, the index has a similar interpretation to the AUC (and consequently, the same range).

Internal Validation In

order to get a sense of how well the 4Dsurvival network would generalize to an external validation cohort, its predictive accuracy was assessed within the training sample using a bootstrap-based procedure recommended in the guidelines for Transparent Reporting of a multivariable model for Individual Prognosis Or Diagnosis (TRIPOD)—see Moons, K. et al. Transparent reporting of a multivariable prediction model for Individual Prognosis Or Diagnosis (TRIPOD): Explanation and elaboration. Ann Intern Med 162, W1-W73 (2015).

This procedure attempts to derive realistic, ‘optimism-adjusted’ estimates of the model's generalization accuracy using the training sample.

(Step 1) A prediction model was developed on the full training sample (size N), utilizing the hyperparameter search procedure discussed above to determine the best set of hyperparameters. Using the optimal hyperparameters, a final model was trained on the full sample. Then the Harrell's concordance index (C) of this model was computed on the full sample, yielding the apparent accuracy, i.e. the inflated accuracy obtained when a model is tested on the same sample on which it was trained/optimized.

(Step 2) A bootstrap sample was generated by carrying out N random selections (with replacement) from the full sample. On this bootstrap sample, a model was developed (applying exactly the same training and hyperparameter search procedure used in Step 1) and computed C for the bootstrap sample (henceforth referred to as bootstrap performance). Then the performance of this bootstrap-derived model on the original data (the full training sample) was also computed (henceforth referred to as test performance)

(Step 3) For each bootstrap sample, the optimism was computed as the difference between the bootstrap performance and the test performance.

(Step 4) Steps 2 to 3 were repeated B times (where B=100).

(Step 5) The optimism estimates derived from Steps 2 to 4 were averaged across the B=100 bootstrap samples and the resulting quantity was subtracted from the apparent predictive accuracy from Step 1.

This procedure yields an optimism-corrected estimate of the model's concordance index:

$${C}_{c o r rected} = {C}_{f u l l}^{f u l l} - \frac{1}{B} {\sum }_{b = 1}^{B} ( {C}_{b}^{b} - {C}_{b}^{f u l l} ) \qquad ( 9 )$$

Above, symbol Cₛ₁^{s1} refers to the concordance index of a model trained on sample s₁ and tested on sample s₂. The first term refers to the apparent predictive accuracy, i.e. the (inflated) concordance index obtained when a model trained on the full sample is then tested on the same sample. The second term is the average optimism (difference between bootstrap performance and test performance) over the B=100 bootstrap samples. It has been demonstrated that this sample-based average is a nearly unbiased estimate of the expected value of the optimism that would be observed in external validation. Subtraction of this optimism estimate from the apparent predictive accuracy gives the optimism-corrected predictive accuracy.

Conventional Parameter Model

As a benchmark comparison to the 4Dsurvival motion model, a Cox proportional hazards model was trained using conventional right ventricular (RV) volumetric indices including right ventricular end-diastolic volume (RVEDV), right ventricular end-systolic volume (RVESV) and the difference between these measures expressed as a percentage of RVEDV, right ventricular ejection fraction (RVEF) as survival predictors. To account for collinearity among these predictor variables, an L₂-norm regularization term was added to the Cox partial likelihood function:

$$log L ( β ) = {\sum }_{n = 1}^{N} {δ}_{n} \{ {β}^{′} {z}_{n} - log {\sum }_{j ϵ R ( {t}_{n} )} exp ( {β}^{′} {z}_{j} ) \} + \frac{1}{2} λ {‖ β ‖}^{2} \qquad ( 10 )$$

in which λ is a parameter that controls the strength of the penalty term. The optimal value of λ was selected via cross-validation.

Interpretation of the 4Dsurvival Model

To facilitate interpretation of the 4Dsurvival network, Laplacian Eigenmaps were used to project the learned latent representations 12 into two dimensions (FIG. 5A), allowing latent space visualization. Neural networks derive predictions through multiple layers of nonlinear transformations on the input data. This complex architecture does not lend itself to straightforward assessment of the relative importance of individual input features. In order to analyse this, a simple regression-based inferential mechanism was used to evaluate the contribution of motion in various regions of the RV to the model's predicted risk (FIG. 5B). For each of the 202 vertices in the time resolved three-dimensional models 4 used in the clinical study, a single summary measure of motion was computed by averaging the displacement magnitudes across 20 frames. This yielded one mean displacement value per vertex. This process was repeated across all subjects. Then the predicted risk scores were regressed onto these vertex-wise mean displacement magnitude measures using a mass univariate approach, i.e. for each vertex v (v=1, . . . , 202), a linear regression model was fitted where the dependent variable was predicted risk score, and the independent variable was average displacement magnitude of vertex v.

Each of these 202 univariate regression models was fitted on all subjects and yielded one regression coefficient representing the effect of motion at a vertex on predicted risk. The absolute values of these coefficients, across all vertices, were then mapped onto a template RV mesh to provide a visualization (FIG. 5B) of the differential contribution of various anatomical regions to predicted risk.

From the results of the clinical study, it may be observed that the generalised methods of the present specification permit learning of meaningful latent representations 12 of cardiac motion, which encode information useful for estimating output data 3 in the form of a predicted time to event for an adverse cardiac event and/or an estimate of risk for an adverse cardiac event.

Modifications

It will be appreciated that many modifications may be made to the embodiments hereinbefore described. Such modifications may involve equivalent and other features which are already known in the design, training and application of machine-learning methods for image processing, and which may be used instead of or in addition to features already described herein. Features of one embodiment may be replaced or supplemented by features of another embodiment.

Although the clinical study presented hereinbefore related to a particular type of heart failure, the methods of the present specification are equally applicable to similar analysis of any other heart condition and/or irregularity. This is expected to be the case because any heart condition will, inherently, have an effect on cardiac motion, and the methods of the present specification have been demonstrated, through the clinical study, to be capable of learning robust and meaningful latent representations of cardiac motion.

The same methods described hereinbefore may be applied to groups of patients experiencing different type of cardiac dysfunction. For example, the methods of the present specification may be applied a training set 5 corresponding to patients with left ventricular failure (also known as dilated cardiomyopathy).

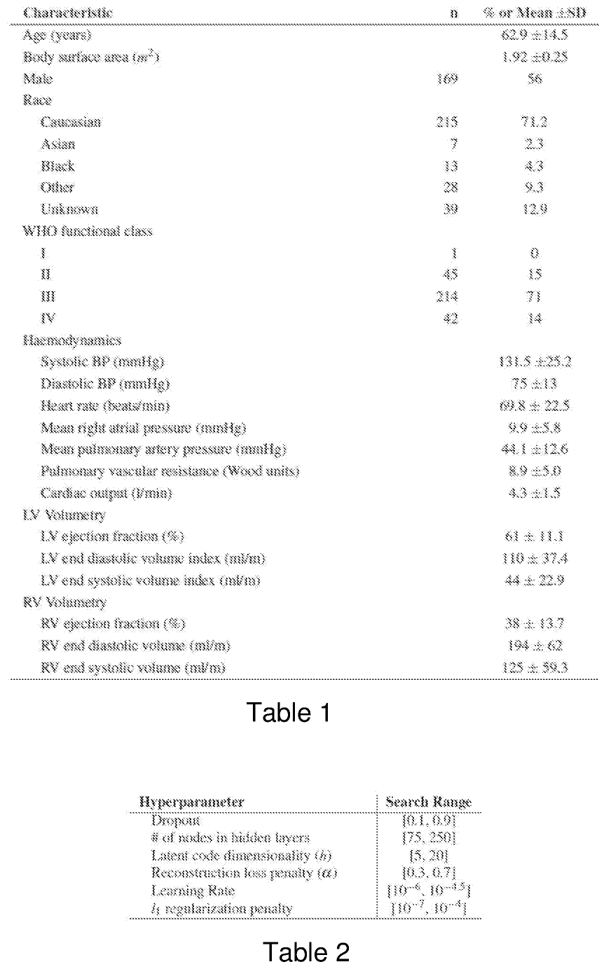

Referring also to FIG. 9, automated segmentation of the left and right ventricles in a patient with left ventricular failure is shown. Referring again to FIG. 3A, further examples of segmenting the left ventricular wall 26 and left ventricular blood pool 28 may be seen (though the data of FIG. 3A relates to patients with pulmonary hypertension rather than left ventricular failure as shown in FIG. 9). The segmented images may be used to create a time-resolved three-dimensional model 4.

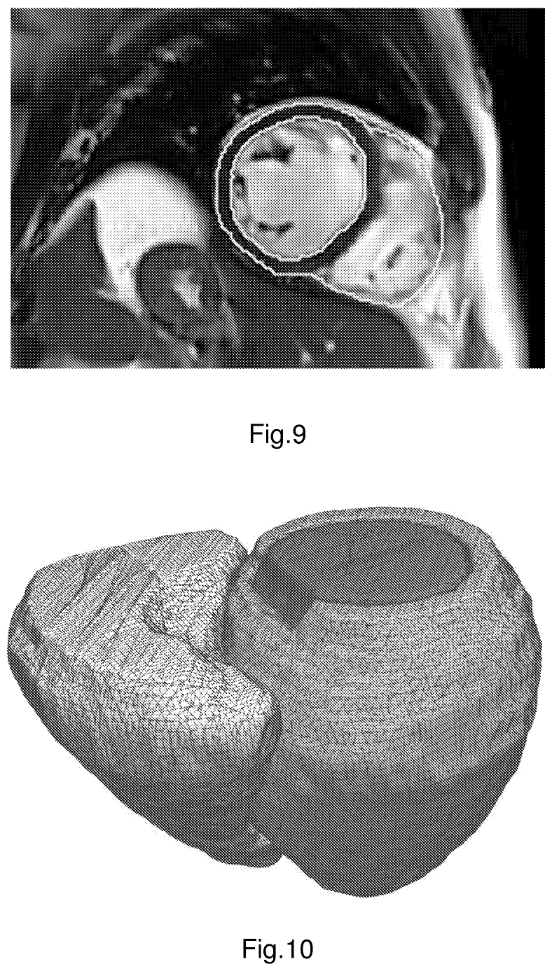

Referring also to FIG. 10, a three-dimensional model of the left and right ventricles describing cardiac motion trajectory is shown for a patient with left ventricular failure.

Such a time-resolved three-dimensional model may be used as input for training a machine learning model, for example the 4Dsurvival network described hereinbefore. The input to the machine learning model 2 may take the form of the time-resolved three-dimensional model 4, or time-resolved trajectories of three-dimensional contraction and relaxation extracted therefrom. The loss function used to the train the machine learning model 2, for example including a reconstruction loss 19 and a prediction loss 20, may be the same as described hereinbefore. Once a trained machine learning model 2 has been obtained, this may be used as described hereinbefore to obtain predictions of outcomes for patients with left ventricular failure (or any other type of cardiac dysfunction).

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicant hereby gives notice that new claims may be formulated to such features and/or combinations of such features during the prosecution of the present application or of any further application derived therefrom.

1. A method of training a machine learning model to:
receive as input a time-resolved three-dimensional model of a heart or a portion of a heart; and
output a predicted time-to-event or a measure of risk for an adverse cardiac event; the method comprising:
receiving a training set which comprises:
a plurality of time-resolved three-dimensional models of a heart or a portion of a heart,
for each time-resolved three-dimensional model, corresponding outcome data associated with the time-resolved three-dimensional model;
using the training set as input, training the machine learning model to recognise latent representations of cardiac motion which are predictive of an adverse cardiac event;
storing the trained machine learning model.

2. A method according to any claim 1, wherein each time-resolved three-dimensional model comprises a plurality of vertices, each vertex comprising a coordinate for each of a plurality of time points;
wherein each time-resolved three-dimensional model is input to the machine learning model as an input vector which comprises, for each vertex, the relative displacement of the vertex at each time point after an initial time point.

3. A method according to claim 1, wherein the machine learning model comprises an encoding layer configured to encode latent representations of cardiac motion.

4. A method according to claim 3, wherein the machine learning model is configured so that the output predicted time-to-event or measure of risk for an adverse cardiac event is determined using a prediction branch which receives as input the latent representation of cardiac motion encoded by the encoding layer.

5. A method according to claim 1, wherein the machine learning model comprises a de-noising autoencoder.

6. A method according to claim 3, wherein the machine learning model is trained according to a hybrid loss function which comprises a weighted sum of:
a first contribution determined based on the input time-resolved three-dimensional models and corresponding reconstructed models of cardiac motion, each reconstructed model determined based on the latent representations of cardiac motion encoded by the encoding layer; and
a second contribution determined based on the outcome data and the corresponding outputs of predicted time-to-event or measure of risk for an adverse cardiac event.

7. A method according to claim 1 wherein training the machine learning model comprises optimising one or more hyperparameters selected from the group consisting of:
a predetermined fraction of inputs to the machine learning model which are set to zero at random;
a number of nodes included in a hidden layer of the machine learning model;
the dimensionality of an encoding layer which encodes a latent representation of cardiac motion;
weights of the first and second contributions to the hybrid loss function;
a learning rate for training the machine learning model; and
an l₁ regularization penalty used for training the machine learning model.

8. A method according to claim 7, wherein optimising one or more hyperparameters comprises particle swarm optimisation.

9. A method according to claim 1, wherein the machine learning model is trained to output a predicted time-to-event or a measure of risk for an adverse cardiac event associated with heart dysfunction.

10. (canceled)

11. A non-transient computer-readable storage medium storing a machine learning model trained to receive as input a time-resolved three-dimensional model of a heart or a portion of a heart, and to output a predicted time-to-event or a measure of risk for an adverse cardiac event.

12. A method comprising:
receiving a time-resolved three-dimensional model of a heart or a portion of a heart;
providing the time-resolved three-dimensional model to a trained machine learning model, the trained machine learning model configured to recognise latent representations of cardiac motion which are predictive of an adverse cardiac event;
obtaining, as output of the trained machine learning model, a predicted time-to-event or a measure of risk for an adverse cardiac event.

13. A method according to claim 12, wherein the time-resolved three-dimensional model comprises a plurality of vertices, each vertex comprising a coordinate for each of a plurality of time points;
wherein the time-resolved three-dimensional model is input to the trained machine learning model as an input vector which comprises, for each vertex, the relative displacement of the vertex at each time point after an initial time point.

14. A method according to claim 12, wherein the trained machine learning model comprises an encoding layer configured to encode a latent representation of cardiac motion.

15. A method according to claim 14, wherein the trained machine learning model is configured so that the output predicted time-to-event or measure of risk for an adverse cardiac event is determined using a prediction branch which receives as input the latent representation of cardiac motion encoded by the encoding layer.

16. A method according to claim 12, wherein the machine learning model further outputs a reconstructed model of cardiac motion.

17. A method according to claim 12, wherein the trained machine learning model comprises a de-noising autoencoder.

18. A method according to claim 12, wherein the trained machine learning model is configured to output a predicted time-to-event or a measure of risk for an adverse cardiac event associated with heart dysfunction.

19. (canceled)

20. A method according to claim 12, further comprising:
obtaining a plurality of images of a heart or a portion of a heart, each image corresponding to a different time or a different point within a cycle of the heart;
generating the time-resolved three-dimensional model of the heart or the portion of the heart by processing the plurality of images using a second machine learning model.

21. A method according to claim 12, wherein the trained machine learning model is a machine learning model trained using steps comprising:
receiving a training set which comprises:
a plurality of time-resolved three-dimensional models of a heart or a portion of a heart,
for each time-resolved three-dimensional model, corresponding outcome data associated with the time-resolved three-dimensional model;
using the training set as input, training the machine learning model to recognise latent representations of cardiac motion which are predictive of an adverse cardiac event.

22. A method according to claim 5, wherein the machine learning model is trained according to a hybrid loss function which comprises a weighted sum of:
a first contribution determined based on the input time-resolved three-dimensional models and corresponding reconstructed models of cardiac motion, each reconstructed model determined based on the latent representations of cardiac motion encoded by the encoding layer; and
a second contribution determined based on the outcome data and the corresponding outputs of predicted time-to-event or measure of risk for an adverse cardiac event.
